(19) United States
(12) Patent Application Publication
(10) Pub. No.: US 2009/0157325 A1
(43) Pub. Date: Jun. 18, 2009

(54) BIOSIGNAL MEASURING APPARATUS

(75) Inventors: Joon Young Jung, Daejeon (KR); Young Sung Kim, Daejeon (KR); Yun Kyung Park, Daejeon (KR); Hyung Jik Lee, Daejeon (KR); Jeun Woo Lee, Daejeon (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon (KR)

(21) Appl. No.: 12/141,279

(22) Filed: Jun. 18, 2008

(30) Foreign Application Priority Data
Dec. 14, 2007 (KR) 10-2007-0131126

(51) Int. Cl. G01N 33/48

(52) U.S. Cl. 702/19

(57) ABSTRACT

A biological signal measuring apparatus includes a first biological signal measuring sensor attached to an earphone used for a mobile phone; and a biological signal receiver for receiving a biological signal from the first biological signal measuring sensor to thereby generate first raw data. The apparatus may further include a second biological signal measuring sensor attached to one of the mobile phone and an adaptor connected to the mobile phone, wherein the biological signal receiver receives a biological signal from the second biological signal measuring sensor to thereby generate second raw data. The apparatus is provided with a controller for detecting whether the mobile phone is used, wherein the controller drives the first and the second biological signal measuring sensor to measure the biological signal when the mobile phone is being used, and stops measuring the biological signals when the use of the mobile phone is finished.

### CROSS-REFERENCE(S) TO RELATED APPLICATIONS

The present invention claims priority of Korean Patent Application No. 10-2007-0131126, filed on Dec. 14, 2007, which is incorporated herein by reference.

### FIELD OF THE INVENTION

The present invention relates to a biological signal measuring apparatus; and, more particularly, to a biological signal sensing apparatus capable of automatically measuring a biological signal of a user using a mobile phone of the user and/or an earphone thereof.

This work was supported by the IT R&D program of MIC/IITA. [2005-S-069-03, Development of Wearable System Using Physiological Signal Processing]

### BACKGROUND OF THE INVENTION

As a conventional technology for measuring a biological signal, disclosed is that the biological signal is measured by a body having a plurality of electrodes and attached to a body of a testee.

Further, disclosed is that a health signal measuring apparatus is mounted on the button of a mobile phone, and thus a biological signal is automatically measured as a user brings the button into contact with his or her body.

In addition, disclosed is that the biological signal of a user is measured by that the user installs an electrocardiogram sensor and a heart pulse sensor at predetermined positions on the portable terminal so as to simultaneously measure the electrocardiogram and heart pulse of the user.

Because these conventional technologies measure the biological signal only when the user consciously intends to measure his or her biological signal, the biological signal of the user cannot be automatically measured while a mobile phone is used.

### SUMMARY OF THE INVENTION

It is, therefore, an object of the present invention to check whether or not a user is using a mobile phone, and unconsciously measure a biological signal of the user.

Another object of the present invention is to attach a sensor capable of measuring the biological signal to a mobile phone or an earphone, and to detect the biological signal of the user who is using the mobile phone or comes into contact with the sensor, so that it can detect the biological signal during daily life anywhere and anytime without inconveniencing the user.

In accordance with the present invention, there is provided a biological signal measuring apparatus including a first biological signal measuring sensor attached to an earphone used for a mobile phone; and a biological signal receiver for receiving a biological signal from the first biological signal measuring sensor to thereby generate first raw data.

The biological signal measuring apparatus may further include a second biological signal measuring sensor attached to one of the mobile phone and an adaptor connected to the mobile phone, wherein the biological signal receiver receives a biological signal from the second biological signal measuring sensor to thereby generate second raw data.

Further, the biological signal measuring apparatus may further include a biological signal processor for generating a processed biological signal using the first and the second raw data generated by the biological signal receiver.

Still further, the biological signal measuring apparatus may further include a controller for operating the first and the second biological signal measuring sensor when the earphone comes into contact with an ear of a user.

Preferably, the controller detects whether or not the mobile phone is used, so that it drives the first and the second biological signal measuring sensor to measure the biological signal when the mobile phone is being used, and stops measuring the biological signals when the use of the mobile phone is finished.

The biological signal measuring apparatus having only first biological signal measuring sensor may further include a biological signal processor for generating a processed biological signal using the first raw data generated by the biological signal receiver.

The biological signal measuring apparatus having the first and the second sensor may further include a biological signal storage for storing at least one of the processed biological signal generated by the biological signal processor and the raw data generated by the biological signal receiver.

The biological signal measuring apparatus may further include a display for displaying at least one of the processed biological signal and the raw data.

The biological signal measuring apparatus may further include a transmitter for transmitting at least one of the processed biological signal and the raw data to one of wired and wireless networks.

The biological signal measuring apparatus having only first sensor may further include a controller for detecting whether or not the mobile phone is used, wherein the controller drives the first biological signal measuring sensor to measure the biological signal when the mobile phone is being used, and stops measuring the biological signals when the use of the mobile phone is finished.

The biological signal measuring apparatus may further include a controller for operating the first biological signal measuring sensor when the earphone comes into contact with an ear of a user.

It is preferable that the earphone is a wireless earphone, which has means for amplifying and filtering the biological signal measured by the first biological signal measuring sensor, and then wirelessly transmitting the biological signal to the mobile phone after an analog-digital conversion (ADC) process.

The wireless earphone may be connected to the mobile phone through ZigBee or Bluetooth.

Further, the earphone may be a wired earphone, which has a biological signal transmission line for transmitting the biological signal measured by the first biological signal measuring sensor and an audio transmission line for transmitting an audio signal.

The biological signal measuring apparatus having only first sensor also may include a biological signal storage, which stores at least one of the processed biological signal generated by the biological signal processor and the raw data generated by the biological signal receiver.

The biological signal measuring apparatus of claim may further include a display for displaying at least one of the processed biological signal and the raw data.

Further, the biological signal measuring apparatus may further include a transmitter for transmitting at least one of the processed biological signal and the raw data to one of wired and wireless networks.

The present invention measures the biological signal when the user uses the mobile phone or wears the earphone, so that it can automatically measures the biological signal, thus being more convenient for the user.

### BRIEF DESCRIPTION OF THE DRAWINGS

The above and other objects and features of the present invention will become apparent from the following description of embodiments given in conjunction with the accompanying drawings, in which:

FIG. 1 illustrates an integrated biological signal measuring apparatus using a mobile phone and/or an earphone thereof in accordance with an embodiment of the present invention;

FIG. 2 illustrates a detachable biological signal measuring apparatus using a mobile phone and/or an earphone thereof in accordance with another embodiment of the present invention;

FIG. 3 illustrates a biological signal measuring apparatus using a mobile phone and a wireless earphone thereof in accordance with still another embodiment of the present invention; and

FIG. 4 is a functional block diagram illustrating a biological signal measuring apparatus using a mobile phone and/or an earphone thereof in accordance with the present invention.

### DETAILED DESCRIPTION OF THE EMBODIMENTS

Hereinafter, embodiments of the present invention will be described in detail with reference to the accompanying drawings so that they can be readily implemented by those skilled in the art. The detailed descriptions of known functions and constructions that unnecessarily obscure the subject matter of the present invention will be avoided herein.

The present invention is designed to unconsciously measure a biological signal using a mobile phone and/or an earphone thereof.

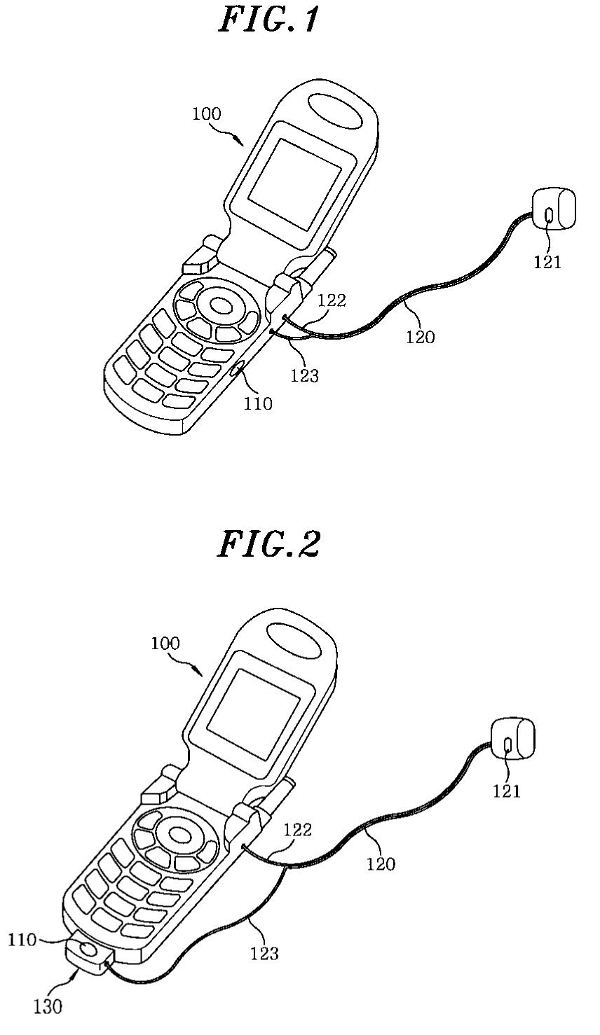

FIG. 1 illustrates an integrated biological signal measuring apparatus using a mobile phone and/or an earphone thereof in accordance with an embodiment of the present invention, in which the biological signal of a user is measured through biological signal measuring sensors 110 and 121 attached to a mobile phone 100 and an earphone thereof 120, respectively.

The biological signal of the user can be measured using the biological signal measuring sensor 110 attached to the mobile phone 100 and/or the biological signal measuring sensor 121 attached to the earphone 120. In other words, the biological signal measuring sensor 110 attached to the mobile phone 100 comes into contact with a hand of the user, and the biological signal measuring sensor 121 attached to the earphone 120 comes into contact with an ear of the user. Thereby, each sensor can detect one-channel electrocardiogram (ECG) signal.

In this manner, in the case of the integrated biological signal measuring apparatus using the mobile phone 100 and/or an earphone thereof 120, since hardware for detecting the biological signal is embedded in the mobile phone 100, the signals measured by the biological signal measuring sensors 110 and 121 are inputted into the mobile phone 100. Particularly, the earphone 120 is provided with an audio transmission line 122 and a biological signal transmission line 123 in order to perform both an ordinary earphone function of the mobile phone 100 and a biological signal measuring function. An audio signal is transmitted through the audio transmission line 122, and the biological signal is transmitted through the biological signal transmission line 123.

Here, examples of the biological signal measuring sensor 110 or 121 include an ECG measuring sensor, a skin temperature (SKT) measuring sensor for detecting the physical temperature of a user, and so on.

An example of measuring an SKT signal of the biological signals will be described. The biological signal measuring apparatus using the mobile phone and/or an earphone thereof measures the biological signal through the biological signal measuring sensor 121 attached to the earphone 120. In detail, the SKT detecting sensor 121 attached to the earphone 120 comes into contact with the ear of the user, thereby measuring the SKT signal. In the case of the biological signal measuring apparatus illustrated in FIG. 1, the hardware for measuring the biological signal is embedded in the mobile phone 100, and the measured biological signal is inputted into the mobile phone 100 through the biological signal transmission line 123.

FIG. 2 illustrates a detachable biological signal measuring apparatus using a mobile phone and/or an earphone thereof in accordance with another embodiment of the present invention.

As illustrated in FIG. 2, the biological signal measuring apparatus in accordance with said another embodiment of the present invention measures a biological signal of a user through biological signal measuring sensors 110 and 121 respectively attached to an adaptor 130 and an earphone 120 which are connected to the mobile phone 100. In other words, the biological signal of the user, for instance ECG or SKT signal, is measured using the biological signal measuring sensor 110 attached to the adaptor 130 and the biological signal measuring sensor 121 attached to the earphone 120.

The biological signal measuring sensor 110 attached to the adaptor 130 comes into contact with the hand of the user, and the biological signal measuring sensor 121 attached to the earphone 120 comes into contact with the ear of the user. Thereby, each sensor measures one-channel ECG signal.

In this manner, in the case of the detachable biological signal measuring apparatus using the mobile phone 100 and/or an earphone thereof 120, since hardware for detecting the biological signal is embedded in the adaptor 130 connected to the mobile phone 100, the signals detected by the biological signal measuring sensors 110 and 121 are inputted into the adaptor 130. Particularly, the earphone 120 includes an audio transmission line 122 and a biological signal transmission line 123 in order to perform an ordinary earphone function of the mobile phone 100 and a biological signal measuring function. An audio signal is transmitted through the audio transmission line 122, and the biological signal is transmitted through the biological signal transmission line 123.

Here, examples of the biological signal measuring sensor 110 or 121 include an ECG measuring sensor, an SKT measuring sensor for measuring the physical temperature of a user, and so on, as described in the first embodiment of the present invention.

An example of measuring an SKT signal of the biological signals will be described. The biological signal measuring apparatus using the mobile phone and/or an earphone thereof detects the biological signal through the biological signal measuring sensor 121 attached to the earphone 120. In detail, the SKT measuring sensor 121 attached to the earphone 120 comes into contact with the ear of the user, thereby measuring the SKT signal. In the case of the biological signal measuring apparatus illustrated in FIG. 2, the hardware for detecting the biological signal is embedded in the adaptor 130, and thus the measurerd biological signal is inputted into the adaptor 130 through the biological signal transmission line 123.

FIG. 3 illustrates a biological signal measuring apparatus using a mobile phone and/or a wireless earphone thereof in accordance with still a third embodiment of the present invention.

As illustrated in FIG. 3, the biological signal measuring apparatus in accordance with said still another embodiment of the present invention measures a biological signal of a user through a biological signal measuring sensor 121 attached to a wireless earphone 140. The wireless earphone 140 is connected to the mobile phone 100 using a wireless communication standard such as Bluetooth or ZigBee.

The biological signal measuring sensor 121 attached to the wireless earphone 140 comes into contact with the ear of the user, thereby measuring the biological signal.

In the case of the biological signal measuring apparatus using the mobile phone 100 and its wireless earphone 140, the biological signal measured by the biological signal measuring sensor 121 is amplified and filtered by the wireless earphone 140, and is transmitted to the mobile phone 100 after being converted into raw data for the biological signal through an analog-digital conversion (ADC) process.

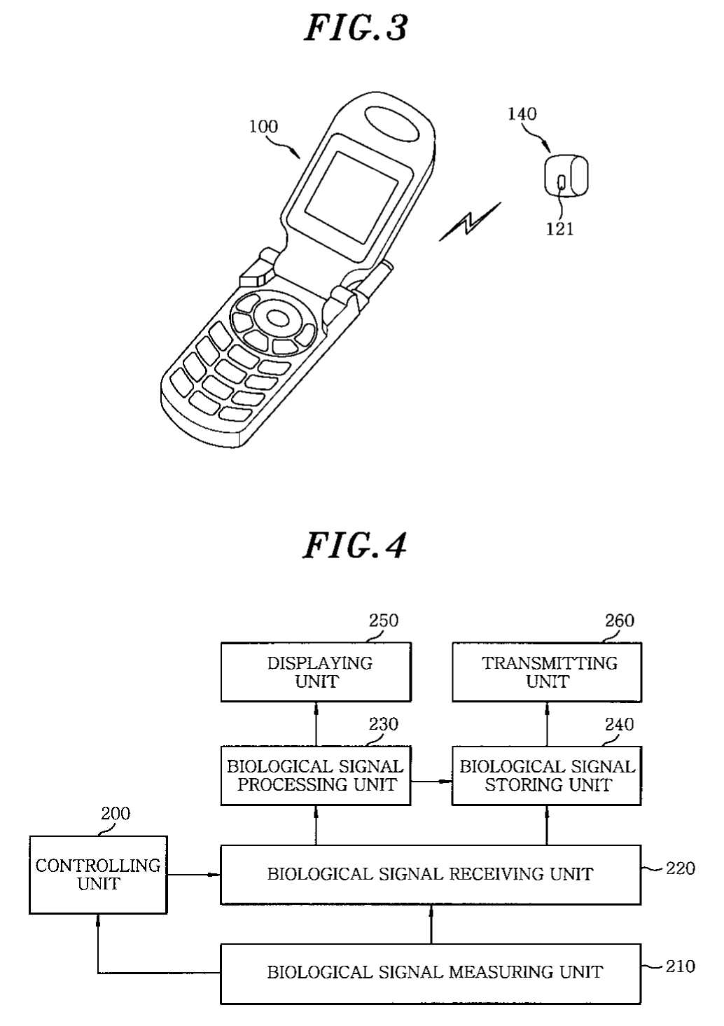

FIG. 4 is a functional block diagram illustrating a biological signal measuring apparatus using a mobile phone and/or an earphone thereof in accordance with the present invention. The biological signal measuring apparatus includes a controlling unit 200, a biological signal measuring unit 210, a biological signal receiving unit 220, a biological signal processing unit 230, a biological signal storing unit 240, a displaying unit 250 and a transmitting unit 250.

The controlling unit 200 determines whether or not the mobile phone 100 is used, or whether or not the biological signal measuring sensors 110 and 121 are worn, and controls the operation of the biological signal measuring apparatus on the basis of the determined result. In other words, when the user uses the mobile phone 100 or comes into contact with the biological signal measuring sensor 110 or 121, the controlling unit 200 detects this situation and starts to measure a biological signal. In contrast, when the user stops using the mobile phone 100 or is separated from the biological signal measuring sensor 110 or 121, the controlling unit 200 detects this situation and stops detecting the biological signal.

The biological signal measuring unit 210 includes the biological signal measuring sensors 110 and 121 attached to the mobile phone 100 or the adaptor 130, and the earphone 120 or the wireless earphone 140, and measures the biological signals, i.e. the ECG or SKT signal.

The biological signal receiving unit 220 amplifies and filters the biological signal inputted from the biological signal measuring unit 210, generates raw data for the biological signal through an ADC process, and transmits the generated raw data to the biological signal processing unit 230.

The biological signal processing unit 230 generates a processed biological signal, such as a heart rate, using the raw data, and then displays the generated biological signal through the displaying unit 250. Thereby, the user can look at his or her biological signal.

The biological signal storing unit 240 stores the raw data generated by the biological signal receiving unit 220 and the processed biological signal generated by the biological signal processing unit 230.

The transmitting unit 260 transmits the biological signals of the user, for instance the raw data or the processed biological signal, which are stored in the biological signal storing unit 240, to another place, such as a hospital's server, a server capable of providing biological signal analysis service, or the like through a network in order to provide various types of healthcare service.

In the case of the biological signal measuring apparatus as illustrated in FIGS. 1 and 2, the controlling unit 200, biological signal measuring unit 210, biological signal receiving unit 220, the biological signal processing unit 230, biological signal storing unit 240, displaying unit 250, and transmitting unit 250 are mounted in the mobile phone 100 or the adaptor installed on the mobile phone. In the case of the biological signal measuring apparatus as illustrated in FIG. 3, the biological signal receiving unit 220 can be mounted in the wireless earphone 140.

As apparent from the above description, the biological signal measuring apparatus in accordance with the present invention measures and store the biological signal when the user uses the mobile phone or wears the earphone, and then transmits the biological signal to the hospital's server, the server capable of providing the biological signal analysis service, or the like through a wired or wireless network, so that, consequently, it can automatically measure the biological signal.

While the invention has been shown and described with respect to the embodiments, it will be understood by those skilled in the art that various changes and modifications may be made without departing from the scope of the invention as defined in the following claims.

What is claimed is:

1. A biological signal measuring apparatus comprising:
a first biological signal measuring sensor attached to an earphone used for a mobile phone; and
a biological signal receiver for receiving a biological signal from the first biological signal measuring sensor to thereby generate first raw data.

2. The biological signal measuring apparatus of claim 1, further comprising:
a second biological signal measuring sensor attached to one of the mobile phone and an adaptor connected to the mobile phone,
wherein the biological signal receiver receives a biological signal from the second biological signal measuring sensor to thereby generate second raw data.

3. The biological signal measuring apparatus of claim 2, further comprising:
a biological signal processor for generating a processed biological signal using the first and the second raw data generated by the biological signal receiver.

4. The biological signal measuring apparatus of claim 2, further comprising:
a controller for operating the first and the second biological signal measuring sensor when the earphone comes into contact with an ear of a user.

5. The biological signal measuring apparatus of claim 2, further comprising:
a controller for detecting whether or not the mobile phone is used,
wherein the controller drives the first and the second biological signal measuring sensor to measure the biological signal when the mobile phone is being used, and stops measuring the biological signals when the use of the mobile phone is finished.

6. The biological signal measuring apparatus of claim 1, further comprising:
a biological signal processor for generating a processed biological signal using the first raw data generated by the biological signal receiver.

7. The biological signal measuring apparatus of claim 3, further comprising:
a biological signal storage for storing at least one of the processed biological signal generated by the biological signal processor and the raw data generated by the biological signal receiver.

8. The biological signal measuring apparatus of claim 7, further comprising:
a display for displaying at least one of the processed biological signal and the raw data.

9. The biological signal measuring apparatus of claim 8, further comprising:
a transmitter for transmitting at least one of the processed biological signal and the raw data to one of wired and wireless networks.

10. The biological signal measuring apparatus of claim 1, further comprising:
a controller for detecting whether or not the mobile phone is used,
wherein the controller drives the first biological signal measuring sensor to measure the biological signal when the mobile phone is being used, and stops measuring the biological signals when the use of the mobile phone is finished.

11. The biological signal measuring apparatus of claim 1, further comprising:
a controller for operating the first biological signal measuring sensor when the earphone comes into contact with an ear of a user.

12. The biological signal measuring apparatus of claim 1, wherein the earphone is a wireless earphone, which has means for amplifying and filtering the biological signal measured by the first biological signal measuring sensor, and then wirelessly transmitting the biological signal to the mobile phone after an analog-digital conversion (ADC) process.

13. The biological signal measuring apparatus of claim 12, wherein the wireless earphone is connected to the mobile phone through ZigBee or Bluetooth.

14. The biological signal measuring apparatus of claim 1, wherein the earphone is a wired earphone, which has a biological signal transmission line for transmitting the biological signal measured by the first biological signal measuring sensor and an audio transmission line for transmitting an audio signal.

15. The biological signal measuring apparatus of claim 6, further comprising:
a biological signal storage, which stores at least one of the processed biological signal generated by the biological signal processor and the raw data generated by the biological signal receiver.

16. The biological signal measuring apparatus of claim 15, further comprising:
a display for displaying at least one of the processed biological signal and the raw data.

17. The biological signal measuring apparatus of claim 16, further comprising:
a transmitter for transmitting at least one of the processed biological signal and the raw data to one of wired and wireless networks.
